(19) United States
(12) Patent Application Publication
(10) Pub. No.: US 2019/0266384 A1
(43) Pub. Date: Aug. 29, 2019

(54) IMAGE PROCESSING SYSTEM FOR OVULATION PREDICTION ANALYSIS

(71) Applicant: ManiGene Co., Ltd., Seoul (KR)

(72) Inventor: Jae Min LEE, Seoul (KR)

(73) Assignee: ManiGene Co., Ltd., Seoul (KR)

(21) Appl. No.: 16/346,016

(22) PCT Filed: Nov. 23, 2016
(86) PCT No.: PCT/KR2016/013569

§ 371 Date: Apr. 29, 2019

(30) Foreign Application Priority Data
Oct. 28, 2016 (KR) 10-2016-0142159
Nov. 23, 2016 (KR) 10-2016-0156484
Nov. 23, 2016 (KR) 10-2016-0156485
Nov. 23, 2016 (KR) 10-2016-0156486

(51) Int. Cl. G06K 9/00; G06T 7/00; G06T 7/62; G01N 21/84; G01N 33/487

(52) U.S. Cl. CPC G06K 9/00147; G06T 7/0012; G06T 7/62; G06T 2207/20021; G06K 9/0014; G01N 21/84; G01N 33/4875; G06K 9/00134

(57) ABSTRACT

Provided is an image processing system for predictive analyzing ovulation, the image processing system includes: an electronic device including a light source and a camera portion; a saliva acquisition device; an image capturing device including a body portion and a light guide member, the body portion being attached to/detached from the electronic device, and the light guide member being arranged in the body portion to guide light and evenly irradiate the light to the saliva acquisition device; and a controller configured to obtain a captured image of the saliva, to extract a crystal structure image of hormone included in the saliva by analyzing the captured image, and to determine whether the object is ovulating based on the crystal structure image.

### TECHNICAL FIELD

The present disclosure relates to an image processing system for ovulation predictive analysis.

### BACKGROUND ART

As society has changed, planning for pregnancy or for contraception has been considered to be important. In case of contraception, natural contraception considering women's ovulation cycle, and artificial contraception using a specified medicine or tool are representative. In case of pregnancy, a method of planning sexual intercourse in consideration of ovulation cycle of women, a method of artificial in vitro/in vivo fertilization through surgical operation, etc. are widely applied. Although high possibility of contraception and pregnancy may exhibit according to the artificial method, the artificial method may adversely affect women's or men's body, and thus, the natural way of taking into account the ovulation cycle of women has been encouraged to use.

A method of measuring ovulation status of a woman may be classified as various types according to parameters for determining whether the ovulation is made, and a method of measuring a content of Lutein hormone in body fluid of a human body and determining whether the ovulation is made is the most accurate. However, the above method requires special safety rules, and there is inconvenience that a test request has to be offered to a specialist of a designated institution and women are not willing to use the above method.

A method of determining the ovulation based on a basal body temperature or a method of collecting mucus from a throat and determining the ovulation after observing crystals during drying the mucus is very convenient to perform and many women use the above method. However, crystals of mucus and the basal body temperature of a woman may change depending on external causes such as diseases or changes in peripheral environment, and thus, reliability in the determination whether the ovulation is made greatly degrades.

### DESCRIPTION OF EMBODIMENTS

### Technical Problem

Provided is an image processing system for predictive analyzing ovulation date through an image analyzation of saliva.

### Solution to Problem

According to an aspect of the present disclosure, an image processing system for predictive analyzing ovulation, the image processing system includes: an electronic device including a light source and a camera portion; a saliva acquisition device including a saliva accommodation portion for accommodating saliva of a subject; an image capturing device including a body portion and a light guide member, the body portion being attached to/detached from the electronic device and including an opening penetrating therethrough in a state in which the saliva acquisition device is mounted to a surface of the body portion and an opposite surface faces the electronic device, and the light guide member being arranged in the body portion to guide light from the light source of the electronic device and evenly irradiate the light from the surface of the body portion to the saliva acquisition device; and a controller configured to obtain a captured image of the saliva from the camera portion, to extract a crystal structure image of hormone included in the saliva by analyzing the captured image, and to determine whether the subject is ovulating based on the crystal structure image.

### Advantageous Effects of Disclosure

According to an image processing system for predictive analyzing ovulation date of one or more embodiments, a captured image of saliva is obtained through a saliva acquisition device and a crystal structure image of hormone is extracted from the captured image and analyzed, and thus, an ovulation may be predicted accurately. Also, the image processing system for predictive analyzing ovulation is portable while storing a plurality of saliva acquisition devices in a storage member. Also, ovulation prediction may be made in a hygienic way through the plurality of saliva acquisition devices, and ovulation may be predicted accurately.

### BRIEF DESCRIPTION OF DRAWINGS

FIG. 1 is a conceptual diagram of an image processing system for predictive analyzing ovulation, according to an embodiment;

FIG. 2 is a perspective view of an image capturing device of FIG. 1;

FIG. 3 is a plan view of the image capturing device of FIG. 1;

FIG. 4 is a cross-sectional view taken along line III-III′ of FIG. 3;

FIG. 5 is a perspective view of a saliva acquisition device of FIG. 1;

FIG. 6 is a diagram showing a correlation between a crystal structure of luteinizing hormone (LH) included in saliva and ovulation cycle;

FIGS. 7A to 7C are diagrams showing a crystal structure of LH according to whether ovulation occurs;

FIG. 8 is a flowchart illustrating a control method of a controller in an image processing and analyzing system for measuring ovulation, according to an embodiment;

FIG. 9 is a conceptual diagram of an image processing system for predictive analyzing ovulation, according to another embodiment;

FIG. 10 is a flowchart illustrating a control method in the image processing system of FIG. 9;

FIG. 11 is a conceptual diagram of an image processing system for predictive analyzing ovulation, according to another embodiment;

FIG. 12 is a perspective view of an image capturing device of FIG. 11;

FIG. 13 is a block diagram showing a relation between the image capturing device of FIG. 11 and a saliva acquisition device;

FIG. 14 is a perspective view showing another example of an image capturing device of FIG. 11;

FIG. 15 is a conceptual diagram showing an implementation of mounting the image capturing device of FIG. 14 in an electronic device;

FIG. 16 is a perspective view of a storage member of FIG. 14; and

FIG. 17 is a cross-sectional view of the storage member of FIG. 16.

### BEST MODE

An embodiment of the present disclosure provides an image processing system for ovulation prediction analysis, the image processing system including: an electronic device including a light source and a camera portion; a saliva acquisition device comprising a saliva accommodation portion for accommodating saliva of a subject; an image capturing device comprising a body portion and a light guide member, the body portion being attached to or detached from the electronic device and comprising an opening penetrating therethrough in a state in which the saliva acquisition device is mounted to one surface of the body portion and an opposite surface faces the electronic device, and the light guide member being arranged in the body portion to guide light from the light source of the electronic device and evenly irradiate the light from the one surface of the body portion to the saliva acquisition device; and a controller configured to obtain a captured image of the saliva from the camera portion, to extract a crystal structure image of hormone included in the saliva by analyzing the captured image, and to determine whether the subject is on an ovulation day based on the crystal structure image.

In an embodiment of the present disclosure, the controller may extract the crystal structure image from the captured image, by dividing the captured image into a plurality of unit pixels and obtaining numerical value (pixel value) from each of the plurality of unit pixels.

In an embodiment of the present disclosure, the controller may extract a plurality of corner points from the captured image, extracts first pixels having numerical values within a first numerical range based on a numerical value of each of the corner points and being continuously connected from the corner points, and determines whether the extracted first pixels correspond to a crystal structure of the hormone based on the extracted first pixels.

In an embodiment of the present disclosure, the plurality of corner points may be relatively extracted by comparing numerical values between an arbitrary unit pixel and peripheral pixels adjacent to the arbitrary unit pixel in each of a plurality of regions included in the captured image.

In an embodiment of the present disclosure, when the extracted first pixels are connected in a length that is equal to or greater than a first reference value set in advance, the controller may determine the extracted first pixels as the crystal structure of the hormone.

In an embodiment of the present disclosure, the controller may calculate a length or a thickness of each of crystal structure images that are extracted, generate classification data by classifying the extracted crystal structure images based on the length or the thickness, and calculate a probability of being on an ovulation day by using the classification data.

In an embodiment of the present disclosure, the controller may determine whether the crystal structure image is valid by using a directionality of the first pixels.

In an embodiment of the present disclosure, the image capturing device may further include an optical portion comprising one or more lenses for magnifying the captured image of the saliva, the optical portion overlapping the opening.

In an embodiment of the present disclosure, the light guide member of the image capturing device may include a light guide portion, into which light from the light source is introduced, and a light irradiation portion surrounding an outer portion of the opening and evenly irradiating the light introduced from the light guide portion to the saliva acquisition device.

In an embodiment of the present disclosure, the saliva acquisition device may further include a body portion and a fixing portion, the body portion supporting the saliva accommodation portion and the fixing portion being disposed in the body portion and fixing the saliva accommodation portion to the image capturing device.

In an embodiment of the present disclosure, the fixing portion may be fixed to the image capturing device by a magnetic force.

An embodiment of the present disclosure provides an image processing system for ovulation detection analysis, the image processing system includes: an electronic device including a camera portion; a saliva acquisition device including a saliva accommodation portion for accommodating saliva of a subject; an image capturing device including a body portion, a light source, and a light guide member, the body portion being attached to/detached from the electronic device and having the saliva acquisition device mounted on a surface thereof and an opening penetrating from the surface to an opposite surface, the light source being arranged adjacent to the opening and irradiating light, and the light guide member guiding the light from the light source to be evenly irradiated to the saliva acquisition device; and a controller configured to obtain a captured image of the saliva from the camera portion, to extract a crystal structure image of hormone included in the saliva by analyzing the captured image, and to determine whether the subject is ovulating based on the crystal structure image.

In an embodiment of the present disclosure, the controller may extract the crystal structure image from the captured image, by dividing the captured image into a plurality of unit pixels and obtaining numerical value (pixel value) from each of the plurality of unit pixels.

In an embodiment of the present disclosure, the controller may extract a plurality of corner points from the captured image, extract first pixels having numerical values within a first numerical range based on a numerical value of each of the corner points and being continuously connected from the each corner point, and determine whether the first pixels correspond to a crystal structure of the hormone based on the first pixels.

In an embodiment of the present disclosure, the plurality of corner points may be relatively extracted by comparing numerical values of an arbitrary unit pixel and peripheral pixels adjacent to the arbitrary unit pixel in each of a plurality of regions included in the captured image, and the controller determines whether the crystal structure image is valid by using a directionality of the first pixels.

In an embodiment of the present disclosure, when the first pixels are connected to a length that is equal to or greater than a first reference value set in advance, the controller may determine the first pixels as the crystal structure of the hormone, calculate a length or a thickness of each of crystal structure images that are extracted, generates classification data by classifying the crystal structure images based on the length or the thickness, and calculate a probability of ovulating by using the classification data.

In an embodiment of the present disclosure, the light guide member of the image capturing device may include a light guide portion, to which light from the light source is introduced, and a light irradiation portion surrounding an outer portion of the opening and evenly irradiating the light introduced from the light guide portion to the saliva acquisition device.

In an embodiment of the present disclosure, the saliva acquisition device may further include a body portion and a fixing portion, the body portion supporting the saliva accommodation portion and the fixing portion fixing the saliva accommodation portion to the image capturing device.

In an embodiment of the present disclosure, the body portion may include: a first body member, on which the saliva acquisition device is mounted, having the opening, the optical portion, and the light source arranged therein; a support member placed on the electronic device and having an adjustable length to be withdrawn toward a front side of the first body member; and a storage member connected to an end portion of the first body member and containing one or more saliva acquisition devices.

In an embodiment of the present disclosure, the body portion may include: a first body member, to which the saliva acquisition device is mounted, having the opening, the optical portion, and the light source arranged therein, the first body member comprising a support recess having a shape corresponding to a shape of the electronic device and placed on the electronic device; and a storage member connected to an end portion of the first body member, the storage member containing one or more saliva acquisition devices.

Other aspects, features and advantages of the present disclosure will become better understood through the accompanying drawings, the claims and the detailed description.

### MODE OF DISCLOSURE

The exemplary embodiments will be described below in more detail with reference to the accompanying drawings. Those components that are the same or are in correspondence are rendered the same reference numeral regardless of the figure number, and redundant explanations are omitted.

As the present disclosure allows for various changes and numerous embodiments, particular embodiments will be illustrated in the drawings and described in detail in the written description. The attached drawings for illustrating one or more embodiments are referred to in order to gain a sufficient understanding, the merits thereof, and the objectives accomplished by the implementation. However, the embodiments may have different forms and should not be construed as being limited to the descriptions set forth herein.

While such terms as “first,” “second,” etc., may be used to describe various components, such components must not be limited to the above terms. The above terms are used only to distinguish one component from another.

An expression used in the singular encompasses the expression of the plural, unless it has a clearly different meaning in the context.

In the present specification, it is to be understood that the terms such as “including,” “having,” and “comprising” are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

It will be understood that when a unit, region, or component is referred to as being “formed on” another layer, region, or component, it can be directly or indirectly formed on the other layer, region, or component. That is, for example, intervening units, regions, or components may be present.

It will be understood that when an element is referred to as being “connected” or “coupled” to another element, it can be directly connected or coupled to the other element or intervening elements may be present.

It will be further understood that the terms “comprises,” “comprising,” “includes” and/or “including,” when used herein, specify the presence of stated features or components, but do not preclude the presence or addition of one or more other features or components.

Sizes of components in the drawings may be exaggerated for convenience of explanation. In other words, since sizes and thicknesses of components in the drawings are arbitrarily illustrated for convenience of explanation, the following embodiments are not limited thereto.

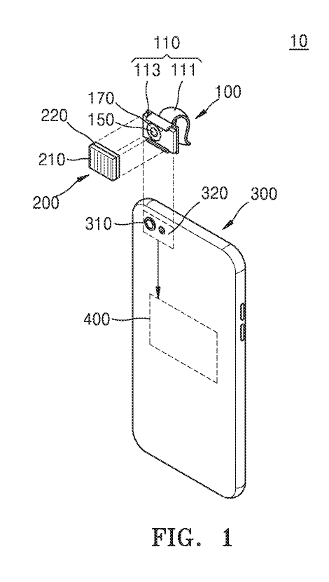

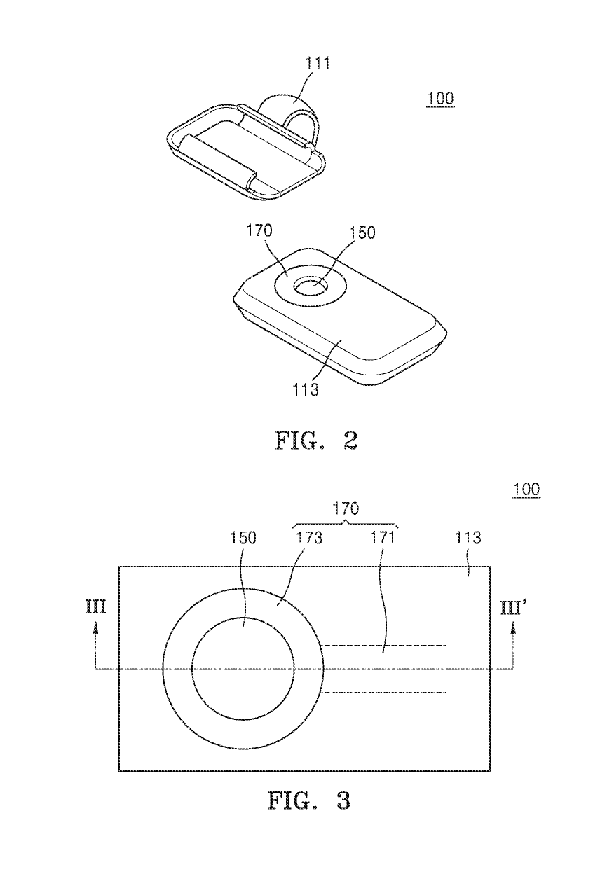

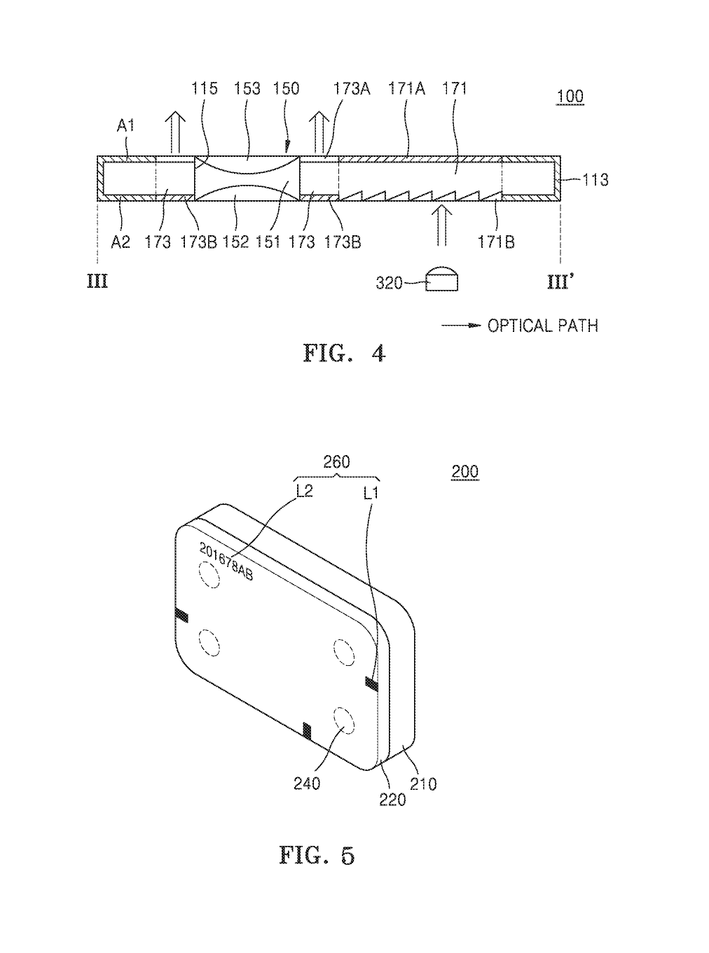

FIG. 1 is a conceptual diagram of an image processing system 10 for predictive analyzing ovulation, according to an embodiment, and FIG. 2 is a perspective view of an image capturing device 100 of FIG. 1. FIG. 3 is a plan view of the image capturing device 100 of FIG. 1, and FIG. 4 is a cross-sectional view taken along line III-III′ of FIG. 3.

Referring to FIG. 1, the image processing system 10 for predictive analyzing ovulation may include the image capturing device 100, a saliva acquisition device 200, an electronic device 300, and a controller 400.

The electronic device 300 may be a portable device including a camera portion 310 and a light source 320 on a surface thereof. The electronic device 300 may include a display (not shown), and the above surface may face a surface in which the display (not shown) is arranged (see FIG. 1), or in another embodiment, the above surface may be equal to the surface in which the display (not shown) is arranged. In another embodiment, the electronic device 300 may include two or more camera portions 310 that may be arranged on both of the surface including the display (not shown) and an opposite surface thereof. For example, the electronic device 300 may be a mobile phone, a tablet, a laptop computer, a graphing calculator, a portable game console, a digital camera, a digital camcorder, a portable media player, etc.

Although not shown in the drawing, the camera portion 310 may include a lens module including at least one lens and an image sensor for sensing an optical image provided to the lens module, to capture an image of the saliva acquisition device 200.

The light source 320 is a light source used in an electronic device such as a general mobile phone and having a wide wavelength band, for example, a light source irradiating light in total wavelength bands of visible ray.

Referring to FIG. 1 to FIG. 4, the image capturing device 100 may be attached to/detached from the electronic device 300, and may include a body portion 110 having the saliva acquisition device 200 mounted on a surface A1 thereof and an opening 115 penetrating through the surface A1 and an opposite surface A2, and a light guide member 170 that guides light from the light source 320 of the electronic device 300 and evenly irradiates the light to the saliva acquisition device 200.

The body portion 110 may be attached to/detached from the electronic device 300. The body portion 110 may include the opening 115 penetrating through the surface A1 and the opposite surface A2. The opening 115 is provided in a location corresponding to the camera portion 310 of the electronic device 300 to expose the camera portion 310. In an embodiment, the body portion 110 may include a support member 111 that may be attached to/detached from the electronic device 300, and a main body 113 coupled to the support member 111. As shown in FIG. 2, the support member 111 may be provided as a clip that may be attached to a side of the electronic device 300. The main body 113 may include the opening 115 described above and is mounted to the electronic device 300 in a state of being coupled to the support member 111, and then, the camera portion 310 of the electronic device 300 may face the saliva acquisition device 200. Although the support member 111 and the main body 113 are separated from each other in the drawings, the support member 111 and the main body 113 of the body portion 110 may be integrally provided and mounted on the electronic device 300. The body portion 110 may have various shapes provided that it may be fixed at a location corresponding to the camera portion 310 of the electronic device 300.

The image capturing device 100 may further include an optical portion 150. The optical portion 150 includes one or more lenses for magnifying an image of saliva and may be located overlapping the opening 115. In an embodiment, the optical portion 150 may include a wide angle lens 152 and a macroscopic lens 153, and a fixing member 151 for fixing the lenses. The optical portion 150 may magnify saliva image of the inserted saliva acquisition device 200 by using the wide angle lens 152 and the macroscopic lens 153, and may be fixed in the opening 115 of the body portion 110 to maintain a constant focal distance. A distance between the camera portion 310 of the electronic device 300 and the optical portion 150 and a distance between the optical portion 150 and the saliva acquisition device 200 may be constant. Therefore, the image processing system 10 for predictive analyzing the ovulation may acquire clear captured image of saliva via the image capturing device 100.

The light guide member 170 may be arranged on the body portion 110. The light guide member 170 guides light from the light source 320 of the electronic device 300 to evenly irradiate the light from a surface of the body portion 110 to the saliva acquisition device 200. The light guide member 170 may include a light guide portion 171 and a light irradiation portion 173.

The light from the light source 320 of the electronic device 300 may be incident to the light guide portion 171. As shown in FIGS. 3 and 4, a side of the light guide portion 171 may correspond to the light source 320, and an opposite side of the light guide portion 171 may be connected to the light irradiation portion 173. Also, when the light from the light source 320 is incident to a light receiving surface 171B that is adjacent to the light source 320, the light is reflected by a reflective surface 171A facing the light receiving surface 171B to enter the light irradiation portion 173. Here, the light receiving surface 171B may have a scattering pattern or an irregular or scratched surface so that the light may be multi-directionally diffused as shown in the drawings. The pattern may include a plurality of grooves each having a ‘V’ shape or a plurality of protrusions. Since the light incident from the light source 320 is diffused through the light-receiving surface 171B and reflected by the reflective surface 171A of the light guide portion 171, the light may be guided to the light irradiation portion 173. The reflective surface 171A faces the light-receiving surface 171B of the light guide portion 171, but one or more embodiments are not limited thereto, that is, the surface facing the light-receiving surface 171B may not be necessarily a reflective surface. The light guide portion 171 may guide light by using a total reflection characteristic of acryl.

The light irradiation portion 173 surrounds an outer portion of the opening 115 of the body portion 110 and may evenly irradiate the light incident from the light guide portion 171 to the saliva acquisition device 200. In the drawings, the light irradiation portion 173 has a rounded shape surrounding the outer portion of the opening 115, but is not limited thereto. The light irradiation portion 173 may have a shape corresponding to the outer portion of the opening 115 or may have a different shape independently from the opening 115. The light irradiation portion 173 irradiates the light while surrounding the outer portion of the opening 115 to evenly irradiate the light to the saliva acquisition device 200. The light may be irradiated from the light irradiation portion 173 through an irradiation surface 173A that is adjacent to the saliva. The light introduced from the light guide portion 171 may be irradiated only through the irradiation surface 173A due to a reflective surface 173B that faces the irradiation surface 173A. The irradiation surface 173A may evenly irradiate the light to the saliva acquisition device 200 by including at least a diffusion sheet. The diffusion sheet may include a polycarbonate-based resin only or a mixture of a polycarbonate-based resin and copolyester-based resin, but any kind of material may be used as the diffusion sheet provided that it may diffuse light. The irradiation surface 173A may further include an optical sheet such as a prism sheet, in addition to the diffusion sheet.

When the light is not evenly irradiated to the saliva acquisition device 200, a hormone crystal structure image that will be described later may not be accurately extracted from the captured image of saliva due to unevenness of the light. Since the image capturing device 100 according to the embodiment may evenly irradiate the light to the saliva acquisition device 200 by using the light guide member 170, the hormone crystal structure image may be exactly extracted from the captured image of saliva.

FIG. 5 is a perspective view of the saliva acquisition device 200 of FIG. 1.

Referring to FIG. 1 and FIG. 5, the saliva acquisition device 200 may include a saliva accommodation portion 220 and a body portion 210.

The saliva accommodation portion 220 may accommodate saliva of a subject on a surface thereof. Here, the saliva may include luteinizing hormone (LH), by which ovulation may be predicted. The saliva accommodation portion 220 may include a light transmitting material such as an acryl material or a plastic film. Saliva of a subject may be collected from an upper portion of the tongue or a lower portion of the tongue by using the saliva accommodation portion 220.

The body portion 210 may support the saliva accommodation portion 220. The body portion 210 may include a colored plastic material, but is not limited thereto. The saliva accommodation portion 220 is arranged on a surface of the body portion 210, and an opposite surface of the body portion 210 may include a plurality of grooves or may include a frictional material. A user may easily mount the saliva acquisition device 200 onto the image capturing device 100 via the opposite surface of the body portion 210.

In addition, the saliva acquisition device 200 according to the embodiment may further include a fixing portion 240 and a product information portion 260.

The fixing portion 240 is arranged on the body portion 210 to fix the saliva accommodation portion 220 to the image capturing device 100. The fixing portion 240 may have various shapes that may be fixed on the image capturing device 100. For example, the fixing portion 240 may be a magnet that may be fixed to the image capturing device 100 via a magnetic force. However, one or more embodiments are not limited thereto, that is, in another embodiment, the fixing portion 240 may be formed as a clip that may fix the saliva acquisition device 200 to the image capturing device 100.

The product information portion 260 is arranged on a location that may be photographed by the camera portion 310 of the electronic device 300, and may include at least one of an encrypted pattern L1 and a serial number L2. As shown in the drawings, the product information portion 260 may be formed on a surface of the saliva accommodation portion 220. The encrypted pattern (L1) may include a barcode, a QR code, a character code, a figure code, etc. Alternatively, the encrypted pattern may include a patterned edge structure of the product information portion. It may be checked whether the saliva acquisition device 200 is a genuine product or is located at a normal position through the product information portion 260. In other words, when the saliva acquisition device 200 is fixed on the image capturing device 100, the image capturing device 100 may obtain an image of the product information portion 260, as well as the captured image of saliva, to notify the user of whether the saliva acquisition device 200 is a genuine product of its own company or whether the fixing location of the saliva acquisition device 200 is at a right position. In addition, the product information portion 260 may include a chip such as a radio frequency identification (RFID), an integrated circuit (IC) chip, etc. The image processing system 10 for predictive analyzing the ovulation according to the embodiment may recognize the encrypted pattern through a captured image of the product information portion 260 or may further include a pattern recognition portion (not shown) that recognizes the encrypted pattern. For example, the image processing system 10 may further include a component linked with a reader of an RFID, IC, etc.

In addition, the controller 400 may obtain a captured image of saliva by using the camera portion 310 and processes the captured image to extract a crystal structure image of hormone included in the saliva. The controller 400 may determine whether the subject is ovulating based on the extracted crystal structure image. Here, the controller may include all kinds of devices capable of processing data, e.g., a processor. Here, ‘processor’ may denote, for example, a data processing device built in hardware, and includes a physically structured circuit for executing functions expressed as code or commands included in a program. Examples of the data processing device built into the hardware may include a microprocessor, a central processing unit (CPU), a processor core, a multiprocessor, an application-specific integrated circuit (ASIC), a field programmable gate array (FPGA), etc., but the scope of the present disclosure is not limited thereto.

In an embodiment, the controller 400 may be arranged in the electronic device 300. The controller 400 may implement the above-described operations by driving an application program or an application stored on a memory (not shown) of the electronic device 300. In another embodiment, the controller 400 may be arranged in the image capturing device 100. In this case, the image capturing device 100 may further include a camera so as to process and analyze the image without using the electronic device 300. For convenience of description, hereinafter, a case in which the controller 400 is arranged in the electronic device 300 will be described.

Hereinafter, a control method of the controller 400 in the image processing system 10 for predictive analyzing the ovulation according to the embodiment will be described below.

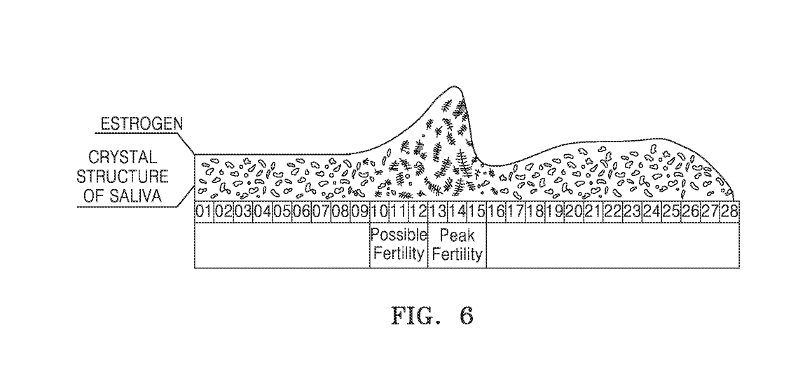

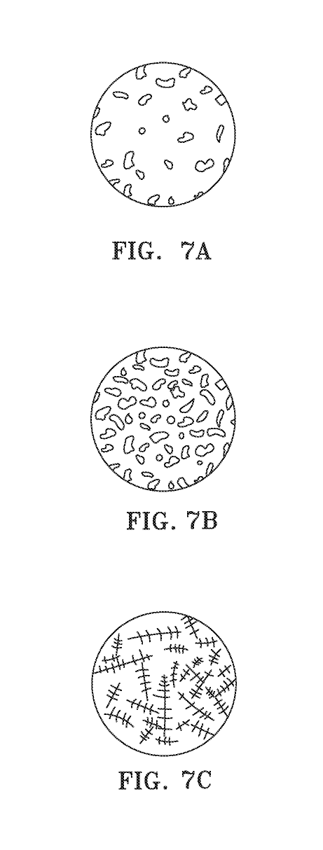

FIG. 6 is a diagram showing a correlation between a crystal structure of LH included in saliva and ovulation cycle, and FIG. 7 (FIGS. 7A to 7C?) is a diagram showing a crystal structure of the LH according to whether the ovulation is made.

Referring to FIGS. 6 and 7, the crystal structure of LH included in saliva changes depending on an ovulation cycle. An infertility period (FIG. 7A), a transition period (FIG. 7B), and an ovulation period (FIG. 7C) may be determined by using above variation in the crystal structure. LH included in saliva may have fern-type crystal structures, in which straight lines cross one another, during the ovulation period. However, even when the saliva has the fern-type crystal structures, it is difficult to determine the ovulation date accurately with the naked eyes. According to the image processing system for predictive analyzing the ovulation of the embodiment, the crystal structure images may be extracted from the captured image of saliva obtained from the camera portion 310 and the crystal structure images are analyzed to provide the user with accurate information about probability of ovulation.

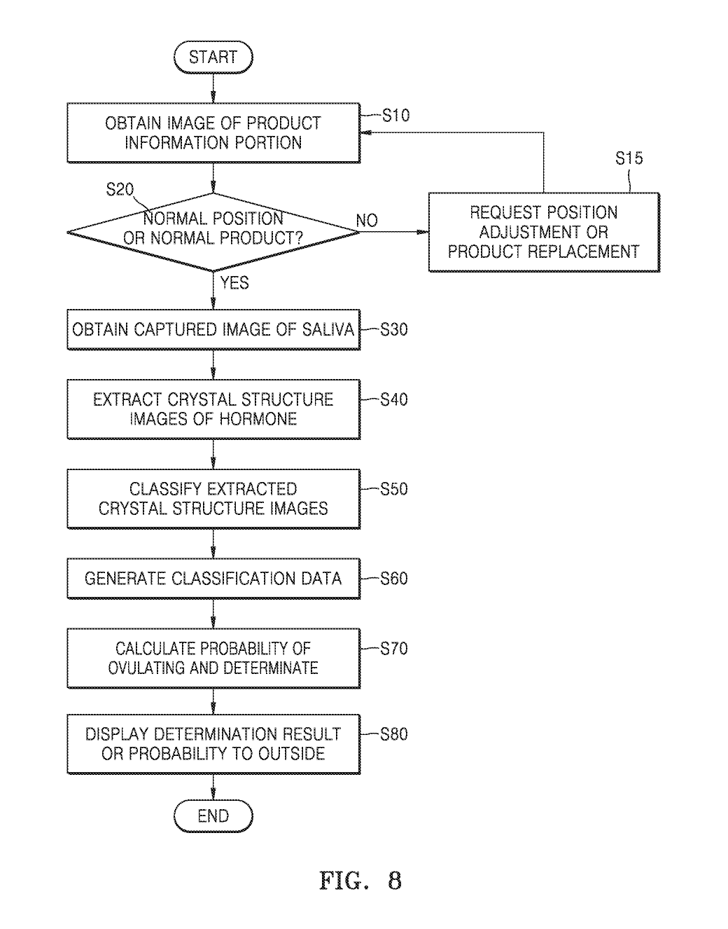

FIG. 8 is a flowchart illustrating a control method of the controller 400 in the image processing system 10 for predicting ovulation, according to an embodiment.

Referring to FIG. 8, the controller 400 may store in advance personal information about a menstrual cycle of a subject and hormone information. When an application is executed on the electronic device 300, a main page including an initial standby page, a calendar screen, an environment setting, etc. may be activated. Here, personal information of the user may be stored on the application for storing periodic or aperiodic data of the user. The personal information or hormone information may be directly input by the user after executing the application, or determination information derived whenever the ovulation is predicted may be stored on a database (not shown) of the controller 400. The personal information of the subject may include height, weight, date of birth, last menstruation date during the last three months, the number of dates of the menstruation, etc.

Next, when the saliva acquisition device 200 is inserted in the image capturing device 100, the controller 400 may obtain an image of the product information portion of the saliva acquisition device 200 (S10). Since the product information portion 260 include at least one of the encrypted pattern and the serial number, the controller 400 may check whether the saliva acquisition device 200 is at a normal position or whether the saliva acquisition device 200 is a genuine product from the obtained image of the product information portion (S20). The controller 400 may also check the number of times that the product is used, a coupling state, and whether the product is cleaned, by using the encrypted pattern or the serial number. The controller 400 may check whether the product is a genuine product by analyzing color of the saliva accommodation portion 220. Here, when the saliva acquisition device 200 is not a genuine product or a normal product or when the position of the saliva acquisition device 200 is not at a normal position, the controller 400 may request the user to replace the saliva acquisition device 200 or to adjust the position of the saliva acquisition device 200 because the saliva acquisition device 200 is at a wrong position (S15).

When the inserted saliva acquisition device 200 is a genuine product and at a normal position, the controller 400 may obtain a captured image of saliva by using the camera portion 310 (S30), and may extract a crystal structure image of hormone included in the saliva by processing the captured image (S40). The captured image of saliva may include foreign matters, as well as the crystal structures. In order to distinguish the crystal structure of hormone from the foreign matters, the controller 400 partitions the captured image as a plurality of unit pixels and digitizes each of the plurality of unit pixels to extract the crystal structure image from the captured image. For example, each of the plurality of unit pixels may be classified as a gray scale having 0 to 255 levels. Here, the controller 400 may relatively extract a plurality of corner points through numerical comparison between an arbitrary unit pixel and peripheral pixels adjacent to the arbitrary unit pixel in each of a plurality of regions included in the captured image. Here, a corner point may be a start point or a finishing point in a lengthwise direction of a hormone crystal structure. In detail, when there is a corner point in a certain region, peripheral pixels adjacent to the corner point may have brighter numerical values as compared with that of the corner point. In other words, when peripheral pixels adjacent to an arbitrary unit pixel have brighter numeral values than that of the arbitrary unit pixel and are continuously connected, the arbitrary unit pixel may be extracted as a corner point.

After that, the controller 400 may extract first pixels having a numerical values within a first numerical range based on the numerical value of the corner point, wherein the first pixels are continuously connected to one another from the corner point. Here, the first pixels may be unit pixels having values similar to the numerical value of the corner point. When the first pixels are continuously connected to one another from the corner point, the controller 400 may determine that the extracted first pixels have the fern-type crystal structure, in which the lines are connected to one another as shown in FIG. 7C.

Here, when the first pixels are connected to one another to a length that is equal to or greater than a first reference value set in advance, the controller 400 may determine the first pixels as a crystal structure of hormone. In other words, even when the first pixels are continuously connected to one another from the corner point, in a case where the connected length is equal to or less than the first reference value, it may not be determined as the fern-type crystal structure corresponding to the ovulation, and the crystal structure image may not be extracted. The first reference value may vary, for example, depending on subjects, and may be determined based on personal information and hormone information of the subject. Also, the controller 400 may determine whether the crystal structure image is valid by using directionality of the first pixels. For example, since the LH of the fern-type crystal structure has an elongated shape, not a circular shape, it may be determined that the crystal structure image is valid in a case where the extracted first pixels have one directionality.

Next, the controller 400 calculates a length or a thickness of each crystal structure image, and classifies extracted crystal structure images based on the length or width (S50). The controller 400 may generate classification date by corresponding each of classification items to the number of crystal structure images corresponding thereto (S60). Referring to FIG. 7C, a plurality of LHs may form a crystal structure in saliva, and a plurality of crystal structures may each have a length and a thickness. The controller 400 may generate classification data by classifying valid crystal structure images by using the length or the thickness. After that, the controller 400 may calculate probability of ovulating by using the classification data (S70). For example, the classification items of the extracted crystal structure images may be graded, and the number of the crystal structure images corresponding to each grade may be digitized to calculate the ovulating probability. In one embodiment, when 15 to 20 first pixels are continuously connected to one another, the controller 400 may classify that crystal structure image as a first grade, and when 10 to 15 first pixels are continuously connected to one another, the controller 400 may classify that crystal structure image as a second grade. The controller 400 may digitize the first grade as 90 points and the second grade as 70 points. The controller 400 may calculate total points throughout the entire captured image by using the value of the grade and the number of crystal structure images included in each grade, and thus, may calculate the probability of ovulating based on the total points. However, the present disclosure is not limited thereto.

The controller 400 may display the calculated probability to outside or may display determined ovulation date to outside (S80). The user may select displaying of probability or displaying of determination result of the ovulation date. The controller 400 may determine that the subject is ovulating when the probability of ovulating is equal to or greater than a reference probability set in advance. Here, the reference probability may be determined based on existing database of the subject or may be arbitrarily selected by the user.

As described above, according to the image processing and analyzing system for predictive analyzing the ovulation and the control method, the captured image of saliva may be clearly obtained by using the image capturing device. Also, the image processing and analyzing system for predictive analyzing the ovulation extracts crystal structure images from the captured image and classifies the crystal structure images to calculate probability, and thus, the ovulation date may be accurately predicted.

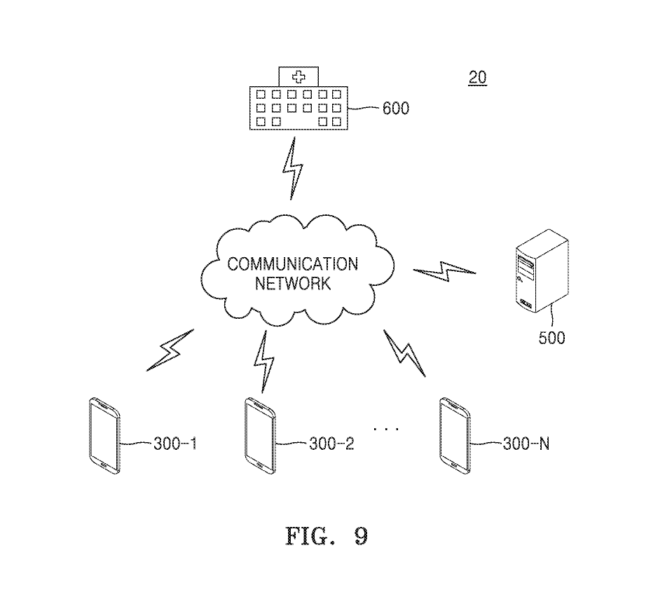

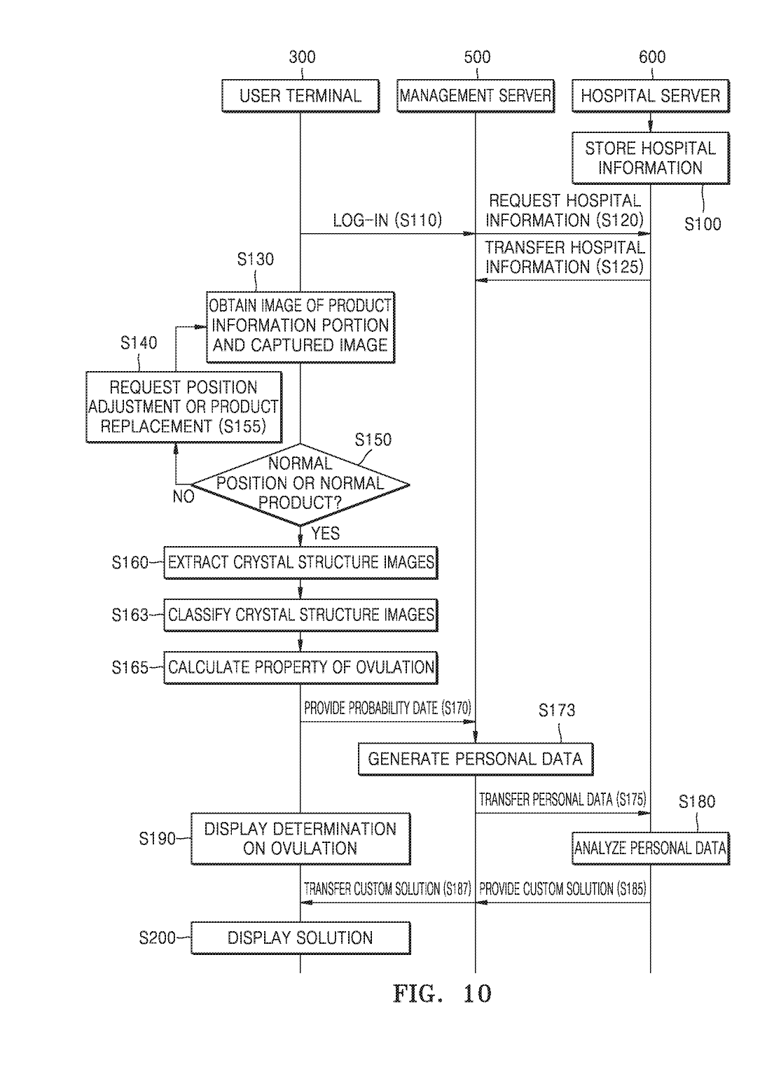

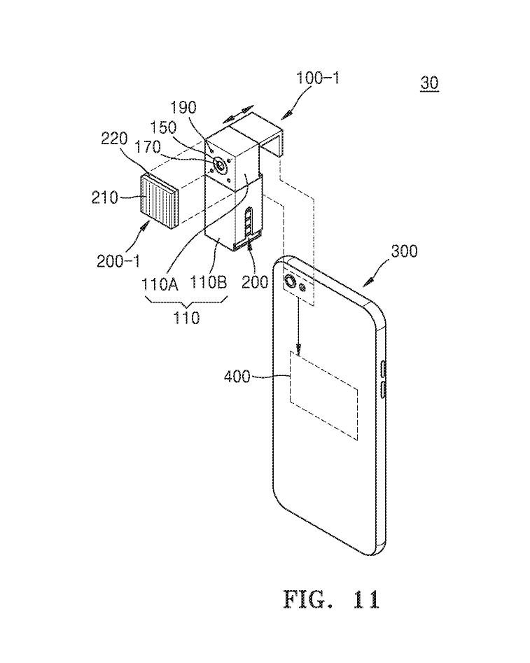

FIG. 9 is a conceptual diagram of an image processing and analyzing system 20 for predicting ovulation according to another embodiment, and FIG. 10 is a flowchart illustrating a control method in the image processing and analyzing system 20 of FIG. 9.

Referring to FIGS. 9 and 10, the image processing and analyzing system 20 for predicting the ovulation according to another embodiment may include an ovulation prediction apparatus (not shown), a saliva acquisition device (not shown), a plurality of electronic devices 300, a management server 500, and a hospital server 600. The image processing and analyzing system 20 for predicting ovulation according to another embodiment may use the ovulation prediction apparatus 100, the saliva acquisition device 200, and the electronic device 300 described above, and may further use the management server 500 and the hospital server 600. In addition, detailed descriptions of the above components are omitted for convenience of description. Here, the controller may be located in each of the plurality of electronic devices 300.

The electronic device 300 may include the camera portion 310. The user collects saliva of a subject by using the saliva acquisition device (not shown), and inserts the saliva acquisition device 200 to the image capturing device to generate a captured image of saliva by using the camera portion 310.

The hospital server 600 may store hospital information including ovulation information, hormone information, etc. of an object measured in a hospital (S100).

The management server 500 may include personal information of a plurality of objects, and the personal information of the plurality of objects may include height, weight, date of birth, last menstruation period in last three months, the number of dates in menstruation cycle, etc.

First, the plurality of electronic devices 300 may include an application connecting to the management server 500 installed thereon, and the user may log-in to the application by executing the application (S110). When a log-in signal is input, the management server 500 may request hospital information of the subject from the hospital server 600 (S120). In the drawings, the user requests the hospital information after logging in, and the hospital server 600 transmits the hospital information (S125). However, one or more embodiments are not limited thereto, that is, the hospital server 600 may transmit the hospital information to the management server 500 in advance so that the management server 500 may store the hospital information in advance.

The user may capture an image of the saliva collected by the saliva acquisition device 200 by using the electronic device 300. Here, the camera portion 310 of the electronic device 300 also captures an image of the product information portion of the saliva acquisition device 200 to obtain the product information portion image and the captured image (S130).

The electronic device 300 may determine whether the saliva acquisition device 200 is a genuine product or the saliva acquisition device 200 is in a normal status by using an encrypted pattern or a serial number in the product information image, or may determine whether the saliva acquisition device 200 is normally inserted (S150). When the saliva acquisition device 200 is at the normal position and in the normal status, the electronic device 300 may extract a crystal structure image by using the captured image (S160). However, when the saliva acquisition device 200 is not at the normal position or not a genuine product, the electronic device 300 may send a message to the user to adjust the position or to replace the product (S140).

When the inserted saliva acquisition device 200 is a genuine product and at the normal position, the electronic device 300 processes the captured image of the saliva to extract crystal structure images of hormone included in the saliva (S160). The electronic device 300 partitions the captured image as a plurality of unit pixels, and digitizes each of the plurality of unit pixels to extract the crystal structure images from the captured image. During the above process, a corner point is extracted, and first pixels having numerical values within a first numerical range based on a numerical value of the corner point and being continuously connected from the corner point may be extracted. The electronic device 300 may extract the crystal structure image by using the first pixels.

Next, the electronic device 300 calculates a length or thickness of each of the extracted crystal structure images, and classifies the extracted crystal structure images based on the length and thickness (S163). The electronic device 300 may generate classification date by corresponding to each of classification items to the number of crystal structure images corresponding thereto. The electronic device 300 may generate classification date by classifying valid crystal structure images corresponding to a reference level by using the length and thickness.

The electronic device 300 may calculate the probability of ovulating by using the classification date (S165). The electronic device 300 may display the calculated probability to outside or may display determined ovulation date to outside (S190). The user may select displaying of probability or displaying of determination result of the ovulation date. The electronic device 300 may determine that the subject is ovulating when the probability of ovulating is equal to or greater than a reference probability set in advance. Here, the reference probability may be determined based on existing database of the subject or may be arbitrarily selected by the user. The electronic device 300 may include a communicator (not shown) capable of communicating with the management server 500, and may transmit probability data including the probability of ovulating generated in the electronic device 300 to the management server 500 via a wireless/wired communicator.

The management server 500 may generate personal data by linking the probability date with the personal information. The management server 500 may store big data including personal data about the plurality of objects. Also, the management server 500 may transmit the personal data to the hospital server 600. Here, the hospital server 600 may analyze the personal data by using the big data stored in the management server (S180). For example, the management server 500 may store the big data, in which personal information of the plurality of subjects including a BMI, menstruation cycle, weight, height, exercise information, etc. is linked with the probability data transmitted from the electronic device 300. The hospital server 600 compares the subject who needs a custom solution with a comparative subject having similar weight and height to those of the subject by using the big data, and may provide a custom solution for improving the probability of predicting the ovulation, e.g., increase in exercise, reduction in BMI, etc. The hospital server 600 may directly transmit the solution to the electronic device 300, or may provide the management server 500 with the custom solution (S185) and make the management server 500 transmit the custom solution to the electronic device 300 (S187). The electronic device 300 may display the custom solution to outside (S200).

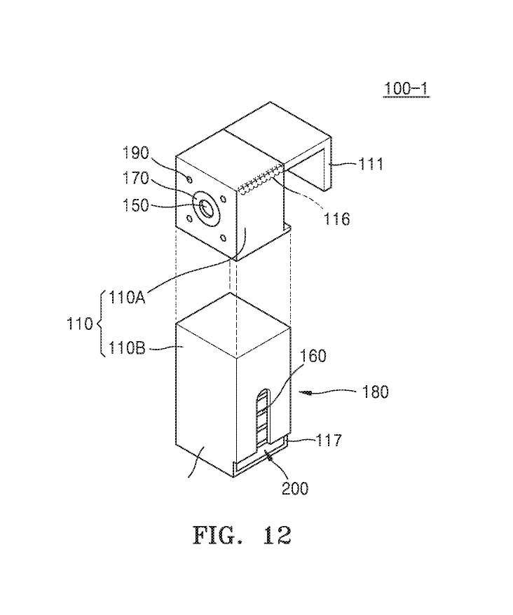

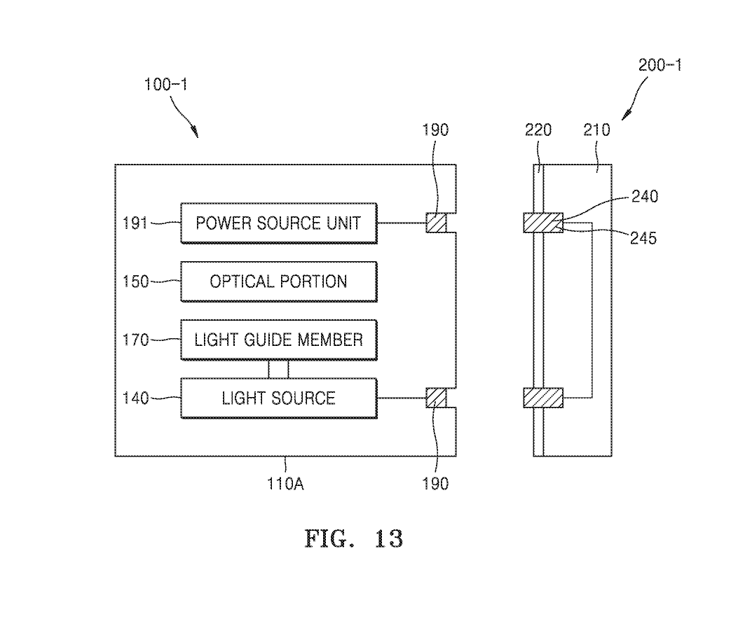

FIG. 11 is a conceptual diagram of an image processing system 30 for predictive analyzing ovulation according to another embodiment, FIG. 12 is a perspective view of an image capturing device 100-1 of FIG. 11, and FIG. 13 is a block diagram showing a relevance between the image capturing device 100-1 and a saliva acquisition device of FIG. 11.

Referring to FIG. 11, the image processing system 30 for predictive analyzing the ovulation according to another embodiment may include the electronic device 300, the image capturing device 100-1, and a saliva acquisition device 200-1. The image processing system 30 according to the another embodiment has the same elements as those of the previous embodiment, except for the image capturing device 100-1, and thus, detailed descriptions about the same elements are omitted and like reference numerals denote the same elements.

The electronic device 300 may be a portable device including a camera portion 310 on a surface thereof. Although not shown in the drawing, the camera portion 310 may include a lens module including at least one lens and an image sensor for sensing an optical image provided to the lens module, to capture an image of the saliva acquisition device 200-1.

Referring to FIGS. 11 and 12, the image capturing device 100-1 may be attached to/detached from the electronic device 300, and may include the body portion 110 having the saliva acquisition device 200 mounted on a surface thereof and an opening penetrating a surface and an opposite surface. Also, the image capturing device 100-1 may include the light source 140 adjacent to the opening to irradiate light, and the light guide member 170 guiding the light from the light source 140 to be irradiated evenly to the saliva acquisition device 200-1.

The light source 140 may be arranged in the body portion 110 to provide the saliva acquisition device 200-1 with light. The light source 140 may be any kind of source that may provide light to the saliva acquisition device 200-1, e.g., a light emitting diode (LED) device.

The image capturing device 100 may further include an optical portion 150. The optical portion 150 includes one or more lenses for magnifying an image of saliva and may be located overlapping the opening 115. A distance between the camera portion 310 of the electronic device 300 and the optical portion 150 and a distance between the optical portion 150 and the saliva acquisition device 200 may be constant.

In addition, the body portion 110 may include a first body member 110A, a support member 111, and a storage member 180.

The first body member 110A has the saliva acquisition device 200-1 mounted on a surface thereof, and may include the opening, the optical portion 150, the light source 140, and the light guide member 170.

The support member 111 has an adjustable length to be withdrawn to a front portion of the first body member 110A, and may be placed on the electronic device 300. The support member 111 may have various shapes that may be placed on the electronic device 300. In the drawings, the support member 111 is arranged on a region of the first body member 110A to be withdrawn to the front portion of the first body member 110A if necessary, and thus, is modified to be placed on the electronic device 300. In this case, when the support member 111 is not placed on the electronic device 300, the support member 111 may be coupled to the first body member 110A so as to improve aesthetic sense and portability. In addition, when the support member 111 is placed on the electronic device 300, the support member 110 may be withdrawn to the front portion of the first body member 110A while adjusting the length thereof, and thus, it may be placed on electronic devices 300 with different thicknesses. A withdrawal length of the support member 111 may be adjusted by an elastic member 116 that is connected to an internal surface of the first body member 110A.

The storage member 180 is connected to an end portion of the first body member 110A and may contain one or more saliva acquisition devices 200. The storage member 180 contains one or more saliva acquisition devices 200, and if necessary, the saliva acquisition device 200 may be withdrawn from the storage member 180 to be used. For example, the storage member 180 may contain ten to twenty saliva acquisition devices 200, and the storage member 180 itself may be replaced after using up the saliva acquisition devices 200. However, one or more embodiments are not limited thereto, and the user may put the saliva acquisition devices 200 in the storage member 180 or take the saliva acquisition devices 200 out from the storage member 180.

The storage member 180 may be attached to/detached from the first body member 110A, and may include a second body member 1106, in which one or more saliva acquisition devices 200 are contained, and an indicator member 160 for indicating the number of saliva acquisition devices 200 to outside. As shown in the drawings, the storage member 180 may be attached to/detached from the first body member 110A via one surface thereof, and it may be checked how many saliva acquisition devices 200 remain in the second body member 1108 through a groove formed in an opposite surface of the storage member 180 in a direction, in which the saliva acquisition devices 200 are stacked. Also, the saliva acquisition device 200 stacked in the second body member 1108 may be easily withdrawn through a withdrawal recess 117 formed in a side of the second body member 1108. Although not shown in the drawings, the storage member 180 may include an elastic unit (not shown) for pushing the stacked saliva acquisition devices 200 in a direction towards the withdrawal recess 117 in order to easily guide the withdrawal of the saliva acquisition devices 200.

The image processing system 30 for predictive analyzing the ovulation according to the embodiment includes the storage member 180 that may contain the plurality of saliva acquisition devices 200, and thus, the image processing system 30 is portable and is capable of predicting the ovulation anywhere.

Referring to FIG. 13, the image capturing device 100-1 may further include a power source unit 191 and a switching unit 190.

The power source unit 191 may supply electric power. The power source unit 191 includes a battery to provide the light source 140 with the electric power. Here, the switching unit 190 may supply the electric power to the light source 140 when the saliva acquisition device 200-1 is mounted. In other words, the power source unit 191 may be electrically connected to the light source 140 as the switching unit 190 interposed therebetween. Thus, when the switching unit 190 is electrically connected to the saliva acquisition device 200-1, the electric power may be supplied to the light source 140.

The saliva acquisition device 200-1 may include a fixing portion 240 coupled to the saliva accommodation portion 220 to fix the saliva accommodation portion 220 to the image capturing device 100-1. The fixing portion 240 may include a magnet that is fixed to the image capturing device by a magnetic force. The saliva acquisition device 200-1 may further include a connecting portion 245 that is electrically connected to the switching unit 190, and in one embodiment, the fixing portion 240 and the connecting portion 245 may be integrally formed with each other. In detail, an end portion of the fixing portion 240 is fixed to the body portion 210 and the other end portion may protrude to outside after penetrating through the saliva accommodation portion 220. The fixing portion 240 of the saliva acquisition device 200-1 may be fixed to the image capturing device 100-1 via a protrusion, as well as the magnetic force. Here, the fixing portion 240 includes a conductive magnet to function as the connecting portion 245 simultaneously. In detail, when the connecting portion 245 of the saliva acquisition device 200-1 is connected to the switching unit 190 of the image capturing device 100-1, the electric power from the power source unit 191 may be supplied to the light source 140. As such, the light may be irradiated from the light source 140 only when the saliva acquisition device 200-1 is mounted.

In addition, the light irradiated from the light source 140 may be evenly supplied to the saliva acquisition device 200-1 via the light guide member 170. The light guide member 170 may be arranged on the body portion 110. The light guide member 170 guides the light from the light source 140 to be evenly irradiated to the saliva acquisition device 200-1. The light guide member 170 may include a light guide portion and a light irradiation portion. The light from the light source 140 may be introduced to the light guide portion. A side of the light guide portion may correspond to the light source 140, and the other side may be connected to the light irradiation portion. The light irradiation portion surrounds an outer portion of the opening 115 of the body portion 110 and may evenly irradiate the light incident from the light guide portion to the saliva acquisition device 200-1.

The product information portion 260 is arranged on a location that may be photographed by the camera portion 310 of the electronic device 300, and may include at least one of an encrypted pattern L1 and a serial number L2.

In addition, the controller 400 may obtain a captured image of saliva by using the camera portion 310 and processes the captured image to extract a crystal structure image of hormone included in the saliva. The controller 400 may determine whether the subject is ovulating based on the extracted crystal structure image.

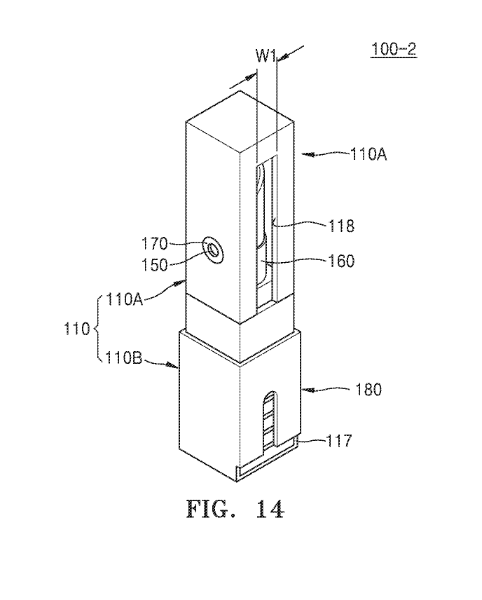

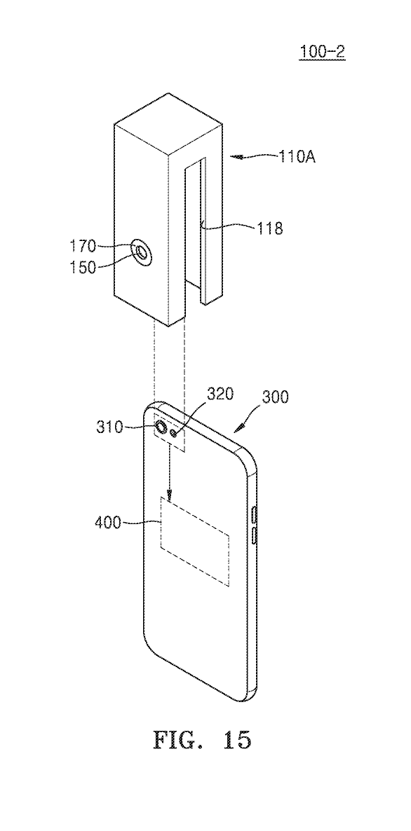

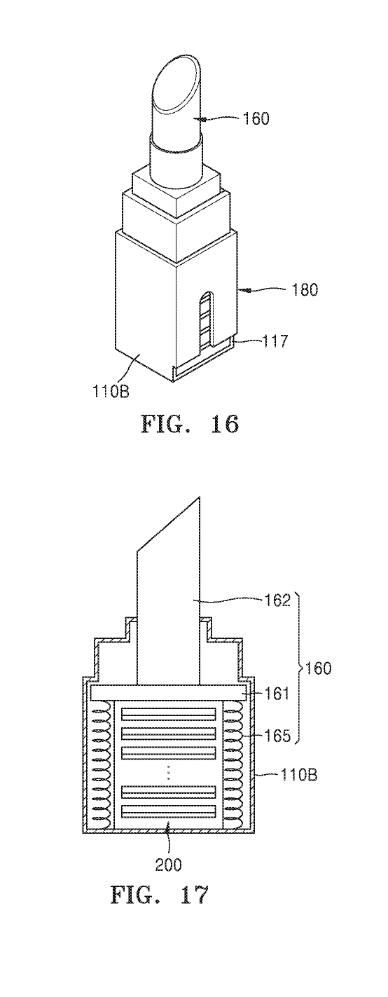

FIG. 14 is a perspective view of an image capturing device 100-2 according to another embodiment, and FIG. 15 is a conceptual diagram showing the image capturing device 100-2 of FIG. 14 mounted on the electronic device 300. FIG. 16 is a perspective view of the storage member 180 of FIG. 14, and FIG. 17 is a cross-sectional view of the storage member 180 of FIG. 16.

Referring to FIGS. 14 and 15, the image capturing device 100-2 may be attached to/detached from the electronic device 300, and may include the body portion 110A having the saliva acquisition device 200 mounted on a surface thereof and an opening penetrating a surface and an opposite surface. Also, the image capturing device 100-2 may include the light source 140 adjacent to the opening to irradiate light, and the light guide member 170 guiding the light from the light source 140 to be irradiated evenly to the saliva acquisition device 200.

The light source 140 may be arranged in the body portion 110 to provide the saliva acquisition device 200 with light. The light source 140 may be any kind of source that may provide light to the saliva acquisition device 200, e.g., a light emitting diode (LED) device.

The image capturing device 100-2 may further include an optical portion 150. The optical portion 150 includes one or more lenses for magnifying an image of saliva and may be located overlapping the opening 115. A distance between the camera portion 310 of the electronic device 300 and the optical portion 150 and a distance between the optical portion 150 and the saliva acquisition device 200 may be constant.

The body portion 110 may include the first body member 110A and the storage member 180.

The first body member 110A has the saliva acquisition device 200 mounted thereon, and may include the opening, the optical portion 150, and the light source 140. Also, the first body member 110A may include a support recess 118 corresponding to a shape of the electronic device 300 so as to be placed on the electronic device 300. As shown in the drawings, the first body member 110A is placed so that the opening or the optical portion 150 may correspond to the camera portion 310 of the electronic device 300, and the support recess 118 may be formed in a surface that extends from the surface where the optical portion 150 is arranged. The first body member 110A may be stably placed on the electronic device 300 via the support recess 118. A width W1 of the support recess 118 may correspond to a thickness of the electronic device 300. In another embodiment, the image capturing device 100-2 may be placed on various electronic devices 300 by inserting an auxiliary unit into the support recess 118 of the width W1 so as to correspond to the thickness of the electronic device 300. Also, the auxiliary unit (not shown) may include a rubber material so that the first body member 110A may be stably fixed on the electronic device 300 without sliding on the electronic device 300.

Referring to FIGS. 16 and 17, the storage member 180 is connected to an end portion of the first body member 110A and may contain one or more saliva acquisition devices 200. As shown in the drawings, the storage member 180 may be connected to the first body portion 110A by being inserted to the end portion of the first body member 110A. The storage member 180 may be attached to/detached from the first body member 110A, and may include a second body member 1106, in which one or more saliva acquisition devices 200 is contained, and an indicator member 160 for indicating the number of saliva acquisition devices 200 to outside. In detail, one or more saliva acquisition devices 200 may be contained in the second body member 1108. For example, the second body member 1108 may contain ten to twenty saliva acquisition devices 200, and the storage member 180 itself may be replaced after using up the saliva acquisition devices 200. The saliva acquisition device 200 stacked in the second body member 1108 may be easily withdrawn through the withdrawal recess 117 formed in a side of the second body member 110B.

The indicator member 160 may include a reference portion 161 and a display portion 162. The reference portion 161 may fix the saliva acquisition devices 200 stacked in the storage member 180 in a direction. In other words, the reference portion 161 is separated from the withdrawal recess 117 of the storage member 180, and one or more saliva acquisition devices 200 may be stacked between the withdrawal recess 117 and the reference portion 161. Here, the reference portion 161 is connected to the elastic unit 165 to retain a fixing force even when the saliva acquisition device 200 is withdrawn through the withdrawal recess 117, so that the saliva acquisition devices 200 may be sequentially withdrawn. The display portion 162 is connected to the reference portion 161 that faces the saliva acquisition devices 200. Also, the display portion 162 may indicate how many saliva acquisition devices 200 remain therein, through a length protruding out of the storage member 180. In other words, when the saliva acquisition devices 200 in the storage member 180 are reduced, a location of the reference portion 161 is switched towards the withdrawal recess 117 due to the elastic unit 165. Here, since the display portion 162 connected to the reference portion 161 is also moved with the reference portion 161, a protruding length of the display portion 162 is reduced, and accordingly, it may be indicated how many saliva acquisition devices 200 remain in the storage member 180. For example, the above type may be provided as a form of a lipstick to apply an aesthetic sense.

As described above, the image processing system for predictive analyzing the ovulation according to the embodiment may be portable while containing the plurality of saliva acquisition devices in the storage member. Also, ovulation prediction may be made in a hygienic way through the plurality of saliva acquisition devices, and ovulation may be predicted accurately.

While the present disclosure has been particularly shown and described with reference to preferred embodiments thereof, it will be understood by those skilled in the art that various changes in form and details may be made therein without departing from the spirit and scope of the present disclosure as defined by the appended claims. The preferred embodiments should be considered in descriptive sense only and not for purposes of limitation. Therefore, the scope of the present disclosure is defined not by the detailed description of the disclosure but by the appended claims, and all differences within the scope will be construed as being included in the present disclosure.

### INDUSTRIAL APPLICABILITY

According to the present disclosure, an image processing system for predictive analyzing ovulation is provided. Also, embodiments of the disclosure may be applied to a hormone analyzing unit.

1. An image processing system for predictive analyzing ovulation, the image processing system comprising:
an electronic device comprising a light source and a camera portion;
a saliva acquisition device comprising a saliva accommodation portion for accommodating saliva of a subject;
an image capturing device comprising a body portion and a light guide member, the body portion being attached to or detached from the electronic device and comprising an opening penetrating therethrough in a state in which the saliva acquisition device is mounted to one surface of the body portion and an opposite surface faces the electronic device, and the light guide member being arranged in the body portion to guide light from the light source of the electronic device and evenly irradiate the light from the one surface of the body portion to the saliva acquisition device; and
a controller configured to obtain a captured image of the saliva from the camera portion, to extract a crystal structure image of hormone included in the saliva by analyzing the captured image, and to determine whether the subject is on an ovulation day based on the crystal structure image.

2. The image processing system of claim 1, wherein the controller extracts the crystal structure image from the captured image, by dividing the captured image into a plurality of unit pixels and obtaining numerical value (pixel value) from each of the plurality of unit pixels.

3. The image processing system of claim 2, wherein the controller extracts a plurality of corner points from the captured image, extracts first pixels having numerical values within a first numerical range based on a numerical value of each of the corner points and being continuously connected from the corner points, and determines whether the extracted first pixels correspond to a crystal structure of the hormone based on the extracted first pixels.

4. The image processing system of claim 3, wherein the plurality of corner points are relatively extracted by comparing numerical values between an arbitrary unit pixel and peripheral pixels adjacent to the arbitrary unit pixel in each of a plurality of regions included in the captured image.

5. The image processing system of claim 3, wherein, when the first pixels are connected in a length that is equal to or greater than a first reference value set in advance, the controller determines the extracted first pixels as the crystal structure of the hormone.

6. The image processing system of claim 3, wherein the controller calculates a length or a thickness of each of crystal structure images that are extracted, generates classification data by classifying the extracted crystal structure images based on the length or the thickness, and calculates a probability of being on an ovulation day by using the classification data.

7. The image processing system of claim 3, wherein the controller determines whether the crystal structure image is valid by using a directionality of the first pixels.

8. The image processing system of claim 1, wherein the image capturing device further comprises an optical portion comprising one or more lenses for magnifying the captured image of the saliva, the optical portion overlapping the opening.

9. The image processing system of claim 1, wherein the light guide member of the image capturing device comprises a light guide portion, into which light from the light source is introduced, and a light irradiation portion surrounding an outer portion of the opening and evenly irradiating the light introduced from the light guide portion to the saliva acquisition device.

10. The image processing system of claim 1, wherein the saliva acquisition device further comprises a body portion and a fixing portion, the body portion supporting the saliva accommodation portion and the fixing portion being disposed in the body portion and fixing the saliva accommodation portion to the image capturing device.

11. The image processing system of claim 10, wherein the fixing portion is fixed to the image capturing device by a magnetic force.

12. An image processing system for predictive analyzing ovulation, the image processing system comprising:
an electronic device comprising a camera portion;
a saliva acquisition device comprising a saliva accommodation portion for accommodating saliva of a subject;
an image capturing device comprising a body portion, a light source, and a light guide member, the body portion being attached to/detached from the electronic device and having the saliva acquisition device mounted on a surface thereof and an opening penetrating from the surface to an opposite surface, the light source being arranged adjacent to the opening and irradiating light, and the light guide member guiding the light from the light source to be evenly irradiated to the saliva acquisition device; and
a controller configured to obtain a captured image of the saliva from the camera portion, to extract a crystal structure image of hormone included in the saliva by analyzing the captured image, and to determine whether the subject is ovulating based on the crystal structure image.

13. The image processing system of claim 12, wherein the controller extracts the crystal structure image from the captured image, by dividing the captured image as a plurality of unit pixels and obtaining numerical value from each of the plurality of unit pixels.

14. The image processing system of claim 13, wherein the controller extracts a plurality of corner points from the captured image, extracts first pixels having numerical values within a first numerical range based on a numerical value of each of the corner points and being continuously connected from the each corner point, and determines whether the first pixels correspond to a crystal structure of the hormone based on the first pixels.

15. The image processing system of claim 14, wherein the plurality of corner points are relatively extracted by comparing numerical values of an arbitrary unit pixel and peripheral pixels adjacent to the arbitrary unit pixel in each of a plurality of regions included in the captured image, and
the controller determines whether the crystal structure image is valid by using a directionality of the first pixels.

16. The image processing system of claim 14, wherein, when the first pixels are connected to a length that is equal to or greater than a first reference value set in advance, the controller determines the first pixels as the crystal structure of the hormone, calculates a length or a thickness of each of crystal structure images that are extracted, generates classification data by classifying the crystal structure images based on the length or the thickness, and calculates a probability of ovulating by using the classification data.

17. The image processing system of claim 12, wherein the light guide member of the image capturing device comprises a light guide portion, to which light from the light source is introduced, and a light irradiation portion surrounding an outer portion of the opening and evenly irradiating the light introduced from the light guide portion to the saliva acquisition device.

18. The image processing system of claim 12, wherein the saliva acquisition device further comprises a body portion and a fixing portion, the body portion supporting the saliva accommodation portion and the fixing portion fixing the saliva accommodation portion to the image capturing device.

19. The image processing system of claim 12, wherein the body portion comprises:
a first body member, on which the saliva acquisition device is mounted, having the opening, the optical portion, and the light source arranged therein;
a support member placed on the electronic device and having an adjustable length to be withdrawn toward a front side of the first body member; and
a storage member connected to an end portion of the first body member and containing one or more saliva acquisition devices.

20. The image processing system of claim 12, wherein the body portion comprises:
a first body member, to which the saliva acquisition device is mounted, having the opening, the optical portion, and the light source arranged therein, the first body member comprising a support recess having a shape corresponding to a shape of the electronic device and placed on the electronic device; and
a storage member connected to an end portion of the first body member, the storage member containing one or more saliva acquisition devices.
